(19) United States
(12) Patent Application Publication
(10) Pub. No.: US 2010/0291534 A1
(43) Pub. Date: Nov. 18, 2010

(54) Methods and Systems for Isolating, Ex Vivo Expanding and Harvesting Hematopoietic Stem Cells

(75) Inventors: Akon Higuchi, Jhongli City (TW); Siou-Ting Yang, Tainan City (TW); Pei-Tsz Li, Kaohsiung City (TW)

Correspondence Address: THOMAS, KAYDEN, HORSTEMEYER & RISLEY, LLP, 600 GALLERIA PARKWAY, S.E., STE 1500, ATLANTA, GA, 30339-5994, US

(73) Assignee: NATIONAL CENTRAL UNIVERSITY, Jhongli City (TW)

(21) Appl. No.: 12/464,210

(22) Filed: May 12, 2009

(51) Int. Cl. A01N 1/02; C12M 1/00; C12N 5/06

(52) U.S. Cl. 435/2; 435/308.1; 435/307.1; 435/297.2; 435/378

(57) ABSTRACT

Disclosed herein are methods and systems for isolating, ex vivo expanding and harvesting hematopoietic stem cells. Methods and systems described herein are easy to use, time-efficient, and allow isolation, ex vivo expansion and harvest of hematopoietic stem cells either batchly or continuously.

### BACKGROUND

1. Field of Invention

This invention in general relates to methods and systems for isolating, ex vivo expanding and harvesting hematopoietic stem cells.

2. Description of Related Art

Stem cells have gained considerable interest as a treatment for a myriad of diseases, conditions, and disabilities because they provide a renewable source of cells and tissues. The main sources of stem cells are the embryonic stem cells and adult stem cells. Embryonic stem cells are derived from embryos, whereas adult stem cells usually reside in very small numbers in each is tissue and have been found in various tissues and organ, including the brain, bone marrow, peripheral blood, blood vessels, skeletal muscle, skin, umbilical cord, adipose tissue, and liver. The use of embryonic stem cells in the treatment of diseases is controversial because of its implications on life. In contrast, adult stem cells pose no ethical dilemma. An advantage of adult stem cells is that the patient's own cells may be expanded in culture and reintroduced into the patient. Further, the use of the patient's own adult stem cells would prevent rejection of the cells by the immune system without having to use immunosuppressive drugs.

Hematopoietic stem cells are the most commonly used adult stem cells in clinical treatment, however, due to the reason that these stem cells are rare in adult tissues and it is difficult to expand their numbers in cell culture, methods of isolating and proliferating adult stem cells in culture are sought, in hope that sufficient number of adult stem cells may be obtained for further practical clinical purpose. Japanese Patent No: H08-69 disclosed a Ficoll-Hypaque method for isolating mononuclear cells from umbilical cord blood, the method was an open procedure and therefore was prone to contamination by bacteria or fungi. Further, approximately 3 hours of time is required for isolating mononuclear cells, before they can be subjected to ex vivo expansion. A labor intensive method was disclosed in Japanese Patent No: 2008-237136, in which hematopoietic CD34⁺ cells were isolated by HES method followed by use of magnetic beads. Similarly, approximately 3 to 5 hours of time is required for isolating hematopoietic CD34⁺ cells, before they can be subjected to ex vivo expansion. Japanese Patent No: H10-84950 disclosed a permeation method, in which red blood cells were permeated through a non-woven fabric coated with hydrophilic copolymer, CD34⁺ hematopoietic stem cells were thus retained by the non-woven fabric, however, the yield is too low to be of any practical usage.

In view of the above, there exists in this art a need of an improved method of isolating and proliferating adult stem cells, particularly, human hematopoietic stem cells, for use in medical treatments including bone marrow transplant.

### SUMMARY

As embodied and broadly described herein, the invention features systems and methods for isolating, ex vivo expanding and harvesting hematopoietic stem cells. The methods and systems described herein are efficient, easy to use and allow isolating, ex vivo expanding and harvesting hematopoietic stem cells either batchly or continuously.

In one aspect, this invention provides systems for isolating, ex vivo expanding and harvesting hematopoietic stem cells.

In one embodiment, the system is a batch-type system, which includes: a filtering chamber comprising a membrane having a pore size ranges from 2 μm to 100 μm; a first inlet for introducing a source of hematopoietic stem cells into the filtering chamber; a second inlet for introducing a washing solution into the lo filtering chamber; and a first outlet for draining the washing solution out of the filtering chamber. In one example, the system further includes a storage chamber for storing the source of hematopoietic stem cells after it has been permeated through the filtering chamber. In another example, the hematopoietic stem cells are retained by the membrane in the filtering chamber, and the membrane with retained hematopoietic stem cells thereon is then taken out form the chamber and subjected to ex vivo culture in a stem-cell culture medium.

In another embodiment, the system is a continuous-type system that includes: a filtering chamber comprising a membrane having a pore size ranges from 2 μm to 100 μm; a first inlet for introducing a source of hematopoietic stem cells into a filtering chamber; a second inlet for introducing a washing solution into the filtering chamber; a third inlet for introducing a stem-cell culture medium into the filtering chamber; a pump for circulating the stem-cell culture medium in the system; a first outlet for draining the washing solution out of the filtering chamber; and a second outlet for collecting the hematopoietic stem cells. In one example, the system further includes a storage chamber for storing the source of hematopoietic stem cells after it has been permeated through the filtering chamber. In another example, the hematopoietic stem cells are continuously harvested from the stem-cell culture medium collected from the second outlet.

In a second aspect, this invention provides methods for isolating, ex vivo expanding and harvesting hematopoietic stem cells by use of the systems of this invention.

In one embodiment, the method is directed to isolating, ex vivo expanding and harvesting hematopoietic stem cells in a batch manner, the method comprises in sequence the steps of:

(a) providing a batch type system for isolating, ex vivo expanding and harvesting hematopoietic stem cells, the system comprises:
a first inlet for introducing a source of hematopoietic stem cells into the filtering chamber;
a second inlet for introducing a washing solution into the filtering chamber; and
a first outlet for draining the washing solution out of the filtering chamber;

(b) introducing a source of hematopoietic stem cells form the first inlet into the filtering chamber;

(c) introducing a washing solution from the second inlet into the filtering chamber; and

(d) culturing the membrane in a stem-cell culture medium to expand the hematopoietic stem cells retained therein.

In one example, the method further comprises: (c1) draining the washing solution out of the filtering chamber from the first outlet after step (c).

In a second embodiment, the method is directed to isolating, ex vivo expanding and harvesting hematopoietic stem cells in a continuous manner, the method comprises in sequence the steps of:

(a) providing a continuous type system for isolating, ex vivo expanding and harvesting hematopoietic stem cells, the system comprises:
a first inlet for introducing a source of hematopoietic stem cells into the filtering chamber;
a second inlet for introducing a washing solution into the filtering chamber;
a third inlet for introducing a stem-cell culture medium into the filtering chamber;
a pump for circulating the stem-cell culture medium in the system;
a first outlet for draining the washing solution out of the filtering chamber; and
   a second outlet for collecting the hematopoietic stem cells;

(b) introducing a source of hematopoietic stem cells form the first inlet into the filtering chamber;

(c) introducing a washing solution from the second inlet into the filtering chamber;

(d) introducing a stem-cell culture medium from the third inlet into the filtering chamber;

(d) activating the pump to circulate the stem-cell culture medium in the system; and

(f) harvesting the hematopoietic stem cells from the stem-cell culture medium collected from the second outlet.

In one example, the method further comprises the steps of: (b1) storing the source of hematopoietic stem cells in a storage chamber after it has been permeated through the filtering chamber in step (b); (c1) draining the washing solution out of the filtering chamber from the first outlet after step (c); and (d1) sampling the hematopoietic stem cells by taking an aliquot of the stem-cell culture medium from the second outlet after step (d).

The details of one or more embodiments of the invention are set forth in the accompanying description below. Other features and advantages of the invention will be apparent from the detail descriptions, and from claims.

It is to be understood that both the foregoing general description and the following detailed description are by examples, and are intended to provide further explanation of the invention as claimed.

### BRIEF DESCRIPTION OF THE DRAWINGS

The accompanying drawings, which are incorporated in and constitute a part of this specification, illustrate several embodiments of the invention and together with the description serve to explain the principles of the invention.

FIG. 1 is a schematic diagram of the batch-type culture system according to one embodiment of the present invention; and

FIG. 2 is a schematic diagram of the continuous-type culture system according to one embodiment of the present invention.

### DETAILED DESCRIPTION

### Definition

As used herein, the term “stem cell” refers to a master cell that can reproduce indefinitely to form the specialized cells of tissues and organs. A stem cell can divide to produce two daughter stem cells, or one daughter stem cell and one progenitor cell, which then proliferates into the tissue's mature, fully formed cells. As used herein, the term “stem cell” refers to multipotent stem cells, in which the term “multipotent cell” refers to a cell that has the capacity to grow into two or more different cell types of the mammalian body within a given tissue or organ. As used herein, “hematopoietic stem cell” refers to the multipotent cell having the ability to grow into mature blood cells, which include, but are not limited to, red blood cells, leucocytes, megakaryocytes, platelets, and T- and B-lymphocytes.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in the respective testing measurements.

It must be noted that as used herein and in the appended claims, the singular forms “a”, “an”, and “the” include plural referents unless the context clearly dictates otherwise.

Methods, techniques, and/or protocols (collectively “methods”) that can be used in the practice of the invention are not limited to the particular examples of these procedures cited throughout the specification but embrace any procedure known in the art for the same purpose. Furthermore, although some methods may be described in a particular context in the specification, their use in the instant invention is not limited to that context.

### Description of the Invention

The practices of this invention are hereinafter described in detail with respect to methods and systems for isolating, ex vivo expanding and harvesting lo hematopoietic stem cells.

### Batch-Type System and Method for Isolating Ex Vivo Expanding and Harvesting Hematopoietic Stem Cells

According to one embodiment of this invention, a batch-type system for isolating, ex vivo expanding and harvesting hematopoietic stem cells comprises: a filtering chamber comprising a membrane having a pore size ranges from 2 μm to 100 μm; a first inlet for introducing a source of hematopoietic stem cells into the filtering chamber; a second inlet for introducing a washing solution into the filtering chamber; and a first outlet for draining the washing solution out of the filtering chamber.

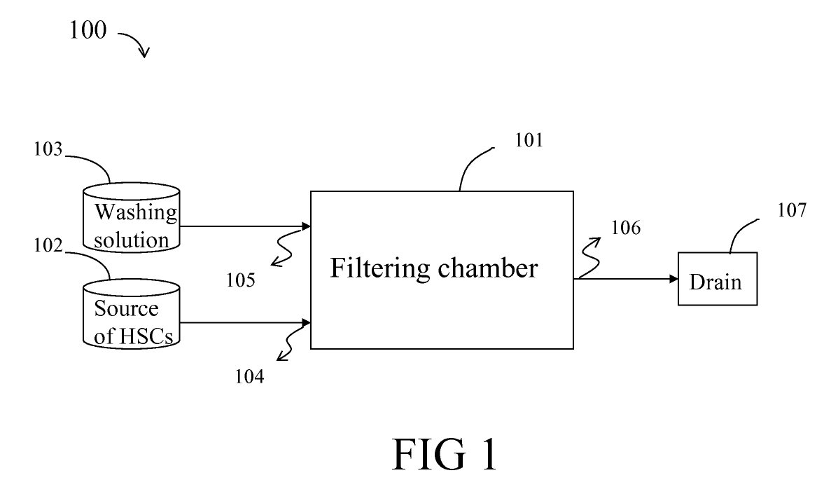

Referring to FIG. 1, which depicts a batch-type system 100 in according to one embodiment of this invention. The system 100 includes a filtering chamber 101, a first inlet 104, a second inlet 105, a first outlet 106, and a drain 107. The filtering chamber 101 comprises a membrane having a pore size ranges from 2 μm to 100 μm, for example, from 2 μm to 25 μm, from 3 μm to 20 μm, or from 3 μm to 15 μm. In operation, a source of hematopoietic stem cells 102 is introduced into the filtering chamber 101 through a first inlet 104; a washing solution 103 is then fed into the filtering chamber 101 through a second inlet 105, and the spent washing solution 103 then exits the filtering chamber 101 through a first outlet 106 into the drain 107. The hematopoietic stem cells are retained by the membrane in the filtering chamber 101, and the entire membrane with retained hematopoietic stem cells therein is then taken out from the filtering chamber 101 and placed in a culturing vessel containing a stem-cell culture medium for ex vivo expansion.

Suitable source of hematopoietic stem cells that may be used in this lo embodiment includes, but is not limited to, a cord blood, a bone marrow aspirates or a peripheral blood. In one example, the source of hematopoietic stem cells is a cord blood collected from a post-partum umbilical cord with informed consent from a woman underwent caesarian procedure or normal birth. In another example, the source of hematopoietic stem cells is a peripheral blood, collected from a qualified donor with informed consent. The cord blood or peripheral blood may be drawn and collected by a syringe and stored in a blood bag contained therein anticoagulants such as citric acids or heparin. In still another example, the source of hematopoietic stem cells is a bone marrow aspirates, collected by the standard bone marrow aspiration protocol with informed consent from a qualified donor. After the source of hematopoietic stem cells 102 has been permeated through the filtering chamber 101, a washing solution 103 is then fed into the system 100 through a second inlet 105. The washing step serves a purpose for further purifying the source of hematopoietic stem cells by removing red blood cells therein, which are believed to have a suppressing effect on the hematopoietic stem cells. Suitable washing solution 103 that may be employed in this embodiment includes, but is not limited to, a serum-free culture medium, a serum-containing culture medium, a saline, a buffer solution, an EDTA containing saline, an EDTA containing buffer solution, a platelet-poor plasma and combinations thereof. The platelet-poor plasma may be produced by any know method in this art using blood as a source. In one example, umbilical cord blood was centrifuged at a suitable speed and the supernatant is then filtered through a membrane filter (0.22 μm in pore size) to remove blood cells therein, and thereby forming the platelet-poor plasma. Alternatively, a culture medium, a saline or a buffer solution may be used as a washing solution 103. The culture medium may or may not contain serum, and the saline or the buffer solution may or may not contain ethylene diamine tetraacetic acid (EDTA). In one example, the washing solution 103 is a serum-free culture medium, for example, the StemSpan SFEM medium purchased directly from StemCell Technologies (USA), which may or may not contain hematopoietic growth factors or other cytokines. In another example, the washing solution 103 is a combination of a serum-free culture medium and a platelet-poor plasma. The source of hematopoietic stem cells or the washing solution may be introduced into the system with an aid of a pump, such as a peristaltic pump. The fluid, either the source of hematopoietic stem cells 102 or the washing solution 103 is fed through the filtering chamber 101 at a flow rate of about 1-10 ml/min. In one example, the flow rate is 1 ml/min.

The filtering chamber 101 comprises a membrane having a pore size ranges from 2 μm to 100 μm within its chamber body. For example, the pore size is between 2 μm to 25 μm, 3 μm to 20 μm, or 3 μm to 15 μm. Several techniques are available for measuring the average pore size of the membranes, such as by scanning electromicroscopy, liquid extrusion porosimetry or other suitable means known in the art. In the illustrated examples, the pore size is estimated by liquid extrusion porosimetry. It is to be noted that the measurement of pore size varies with the particular technique adopted for the measurement. Suitable membrane material should possess good biocompatibility, good moldability, good sterility and low toxicity to the cells. The membrane is generally made from synthetic polymers, which include, but are not limited to, polyethylene, polypropylene, polystyrene, an acrylic resin, nylon, polyester, polycarbonate, polyacrylamide, and polyurethane; natural polymers which include, but are not limited to, agarose, cellulose, cellulose acetate, chitin, chitosan, and alginate; or inorganic materials, which include, but lo are not limited to, hydroxyapatite, glass, alumina, and a titania, and metals such as stainless steel, titanium, and aluminum. Preferably, the membrane is made of a polyurethane-based polymer or a polyethylene terephthalate-based polymer (i.e., non-woven fabric). The base polymer may be further modified by grafting onto its main chain and/or side chain with other molecules. Such molecules include, but are not limited to, amino acids, peptides, glycosaminoglycans, and sugar proteins. In one example, the polyurethane-based polymer is further grafted with functional groups such as carboxylic groups on its surface by a known plasma discharge method, such as the method described in a prior publication, JP 2005-323534, which is hereby incorporated by reference in its entirety. In another example, the polyurethane-based polymer is used directly without the grafted carboxylic groups on its surface. The polyethylene terephthalate-based polymer, on the other hand, may comprise a polymer coating on its surface, the polymer is made from at lease one monomer selected from the group consisting of hydroxyethyl methacrylate (HEMA), dimethylaminoethyl methacrylate (DM), n-butylmethacrylate (BMA), N, N-dimethylacrylamide (DMA), N-acryloylmorpholine (AMO) and N-vinylpyrrolidone (VP). In one example, the membrane is made of polyethylene terephthalate-based polymer, i.e., non-woven fabric, coated with a polymer made from BMA and DMA.

After permeation, the hematopoietic stem cells are retained by the membrane in the filtering chamber 101. Then, the membrane with retained hematopoietic stem cells thereon is taken out form the chamber 101 and carefully placed in a culture vessel containing therein a suitable stem-cell culture medium for direct ex vivo expansion. In one example, the stem-cell culture medium is StemSpan SFEM medium that is further supplemented with a mixture lo of cytokines and low density lipoprotein (LDL). The mixture of cytokines includes a recombinant human stem cell factor, a recombinant human thrombopoietin and a recombinant human Flt-3 ligand. Each cytokine is present in a concentration between 5 ng/ml to 500 ng/ml at the beginning of the culture, and preferably in a concentration between 10 to 100 ng/ml at the beginning of the culture. The use of LDL is optional, and when LDL is included, it is usually present at a dose between 0.1 mg/ml to 20 mg/ml at the beginning of the culture, and preferably at 5 mg/ml at the beginning of the culture. In one example, a serum-free culture medium is used as the washing solution 103 to permeating the filtering chamber 101, the membrane contained thereon retained the hematopoietic stem cells is then subjected to direct ex vivo expansion for about 10 days, and the number of expanded hematopoietic stem cells increases for at least about 63%, as compared with the control, that is, stem cells isolated directed from the source of hematopoietic stem cells and placed into ex vivo expansion. In another example, a platelet-poor plasma is used as the washing solution 103 to permeate the filtering chamber 101, and the number of expanded hematopoietic stem cells increases for at least about 280%, as compared with the control.

The batch-type system of this embodiment is an easy to use and time efficient system for isolating hematopoietic stem cells, the operating time starting from permeating the source of hematopoietic stem cells into the filtering chamber to inoculating the isolated hematopoietic stem cells to culture dishes takes a relatively short period of time, from about 10 min to 60 min. In one preferred embodiment, the operating time is about 18 min.

### Continuous-Type System and Method for Isolating, Ex Vivo Expanding and Harvesting Hematopoietic Stem Cells

According to another embodiment of this invention, a continuous-type system for isolating, ex vivo expanding and harvesting hematopoietic stem cells comprises: a filtering chamber comprising a membrane having a pore size ranges from 2 μm to 100 μm; a first inlet for introducing a source of hematopoietic stem cells into the filtering chamber; a second inlet for introducing a washing solution into the filtering chamber; a third inlet for introducing a stem-cell culture medium into the filtering chamber; a pump for circulating the stem-cell culture medium inside the system; a first outlet for draining the washing solution out of the filtering chamber; and a second outlet for collecting the hematopoietic stem cells.

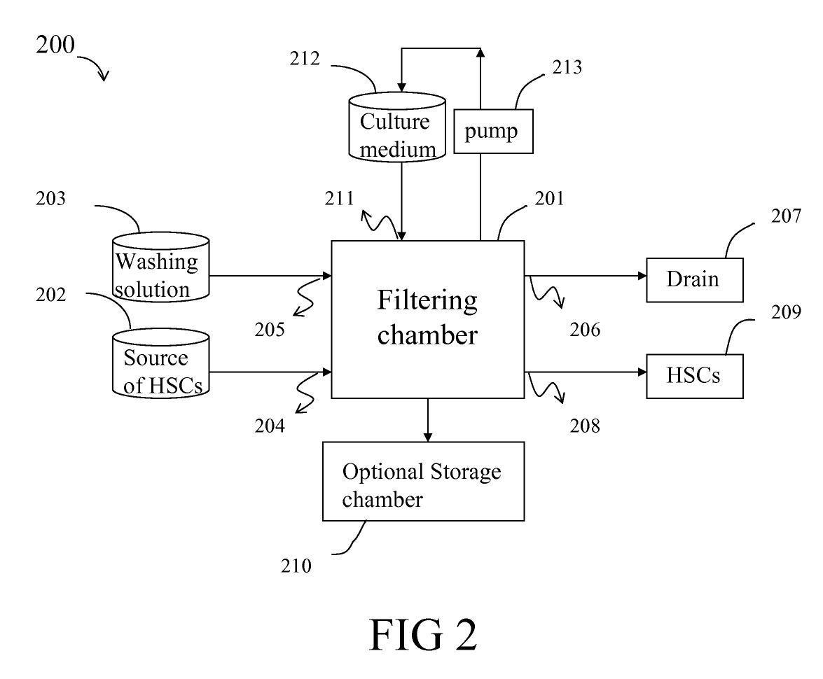

Referring to FIG. 2, which depicts a continuous-type system 200 in according to one embodiment of this invention. The system 200 includes: a filtering chamber 201, a first inlet 204, a second inlet 205, a first outlet 206, a drain 207, a second outlet 208, a hematopoietic stem cell collecting vessel 209, a third inlet 211, and a pump 213. The system may further comprise an optional storage chamber 210 in connection with the filtering chamber 201. The filtering chamber 201 comprises a membrane having a pore size ranges from 2 μm to 100 μm within its chamber body. In operation, a source of hematopoietic stem cells 202 is introduced into the filtering chamber 201 through a first inlet 204. Optionally, the source of hematopoietic stem cells 202 permeated through the filtering chamber 201 are re-collected and stored in the optional storage chamber 210. A washing solution 203 is then fed into the filtering chamber 201 through a second inlet 205, and the spent washing solution 203 then exits the filtering chamber 201 through a first outlet 206 into a drain 207. Subsequently, lo a stem-cell culture medium 212 is fed into the filtering chamber 201 through a third inlet 211 and is further circulated inside the system 200 with an aid of a pump 213. The stem-cell culture medium 212 is replaced every few days, such as every 2 days, by a fresh medium 212, and the spent medium 212 is then exited the system through the first outlet 206 and into the drain 207, while the stem cells remain in a culture state in the filtering chamber 201. Small aliquots of the stem-cell culture medium 212 may also be taken from the second outlet 208 during culture, and the number of hematopoietic stem cells 202 in the medium 212 is analyzed, so as to determine whether enough number of hematopoietic stem cells 202 has been reached. Once the number of hematopoietic stem cells 202 in the sampling medium 212 has reached a desired value, then the medium 212 in the filtering chamber 211 is drained from the second outlet 208 and into the hematopoietic stem cell collecting vessel 209, and hematopoietic stem cells 202 are then harvested from the medium 212 in the collecting vessel 209. Alternatively, the hematopoietic stem cells 202 may also be continuously harvested every few days from the medium 212 collected in the collecting vessel 209 and then pool together for further use. It is to be noted that suitable 2-way or 3-way valves (not shown) are placed in the system to allow formation of separate loops within the system when appropriate. For example, when culture medium 212 is introduced through the third inlet 211, valves located respectively at lines leading to the first inlet 204, the second inlet 205, the drain 207, the collecting vessel 209 and the optional storage chamber 210 are all closed, so that a closed loop among the pump 213, the culture medium 212 and the filtering chamber 204 is formed. Similarly, when aliquots of medium 212 need to be sampled during culturing, then a valve leading to the collecting vessel 209 is opened, while valves leading to the first inlet 204, the second inlet 205, the drain 207, and the optional storage chamber 210 remain closed, so that a loop is formed among the pump 213, the culture medium 212, the filtering chamber 204 and the collecting vessel 209.

Similarly, suitable source of hematopoietic stem cells that may be used in this embodiment includes, but is not limited to, a cord blood, a bone marrow aspirates or a peripheral blood. In one example, the source of hematopoietic stem cells is a cord blood collected from a post-partum umbilical cord with informed consent from a woman underwent caesarian procedure or normal birth. In another example, the source of hematopoietic stem cells is a peripheral blood, collected from a qualified donor with informed consent. The cord blood or peripheral blood may be drawn and collected by a syringe and stored in a blood bag contained therein anticoagulants such as citric acids or heparin. In still another example, the source of hematopoietic stem cells is a bone marrow aspirates, collected by the standard bone marrow aspiration protocol with informed consent from a qualified donor. The source of hematopoietic stem cells 202 are introduced into the filtering chamber 201 through the first inlet 204, and may be re-collected in a storage chamber 210. A washing solution 203 is then fed into the system 200 through a second inlet 205. The introduction of the washing solution 203 serves a purpose of purifying the source of hematopoietic stem cells 202 by removing red blood cells therein, which are believed to have a suppressing effect on the hematopoietic stem cells. Suitable washing solution 203 that may be employed in this embodiment includes, but is not limited to, a serum-free culture medium, a serum-containing culture medium, a saline, a buffer solution, an EDTA containing saline, an EDTA containing buffer solution, a platelet-poor plasma and combinations thereof. The platelet-poor plasma may be produced by any know method in this art using blood as a source. In one lo example, umbilical cord blood was centrifuged at a suitable speed and the supernatant is then filtered through a membrane filter (0.22 μm in pore size) to remove blood cells therein, and thereby forming the platelet-poor plasma. Alternatively, a culture medium, a saline or a buffer solution may be used as a washing solution 203. The culture medium may or may not contain serum, and the saline or the buffer solution may or may not contain ethylene diamine tetraacetic acid (EDTA). In one example, the washing solution 203 is a serum-free culture medium, for example, the StemSpan SFEM medium purchased directly from StemCell Technologies (USA), which may or may not contain hematopoietic growth factors or other cytokines. In another example, the washing solution 203 is a combination of a serum-free culture medium and a platelet-poor plasma. The source of hematopoietic stem cells or the washing solution may be introduced into the system with an aid of a pump (not shown), such as a peristaltic pump. The fluid, either the source of hematopoietic stem cells 202 or the washing solution 203 is fed through the filtering chamber 201 at a flow rate of about 1-10 ml/min.

The filtering chamber 201 comprises a membrane having a pore size ranges from 2 μm to 100 μm within its chamber body. For example, the pore size is between 2 μm to 25 μm, 3 μm to 20 μm, or 3 μm to 15 μm. Several techniques are available for measuring the average pore size of the membrane, such as by scanning electromicroscopy, liquid extrusion porosimetry or other suitable means known in the art. In the illustrated examples, the pore size is estimated by liquid extrusion porosimetry. It is to be noted that the measurement of pore size varies with the particular technique adopted for the measurement. Suitable membrane material should possess good moldability, good biocompatility, good sterility and low toxicity to the cells. The membrane is generally made from synthetic polymers, which include, but are not limited to, polyethylene, polypropylene, polystyrene, an acrylic resin, nylon, polyester, polycarbonate, polyacrylamide, and polyurethane; natural polymers which include, but are not limited to, agarose, cellulose, cellulose acetate, chitin, chitosan, and alginate; or inorganic materials, which include, but are not limited to, hydroxyapatite, glass, alumina, and a titania, and metals such as stainless steel, titanium, and aluminum. Preferably, the membrane is made of a polyurethane-based polymer or a polyethylene terephthalate-based polymer (i.e., non-woven fabric). The base polymer may further be modified by grafting onto its main chain and/or side chain, other molecules. Such molecules include, but are not limited to, amino acids, peptides, glycosaminoglycans, and sugar proteins. In one example, the polyurethane-based polymer is further grafted with functional groups such as carboxylic groups on its surface by a known plasma discharge method, such as the method described in a prior publication, JP 2005-323534, which is hereby incorporated by reference in its entirety. The polyethylene terephthalate-based polymer, on the other hand, may comprise a polymer coating on its surface, the polymer is made from at lease one monomer selected from the group consisting of hydroxyethyl methacrylate (HEMA), dimethylaminoethyl methacrylate (DM), n-butylmethacrylate (BMA), N,N-dimethylacrylamide (DMA), N-acryloylmorpholine (AMO) and N-vinylpyrrolidone (VP). In one example, the membrane is made of polyurethane-based polymer with or without grafted carboxylic groups on the surface. In another example, the membrane is made of polyethylene terephthalate-based polymer, i.e., non-woven fabric, coated with a polymer made from BMA and DMA.

After permeation, some hematopoietic stem cells are retained by the membrane in the filtering chamber 201. For ex vivo expansion, a suitable stem-cell culture medium is introduced into the filtering chamber 201 through the third inlet 211, and circulates therein with the aid of a pump 213. Preferably, the pump 213 is a peristaltic pump, which circulates the culture medium 212 in the system 200, particularly in the filtering chamber 201. The pump 213 may be disposed at various suitable positions as long as a closed loop is formed to circulate the culture medium in the system. In one configuration, a closed loop is formed among the pump 213, the culture medium 212 and the filtering chamber 204 (see FIG. 2) by closing valves located respectively at lines leading to the first inlet 204, the second inlet 205, the optional storage chamber 210, the drain 207, and the second outlet 208. In another configuration, a closed loop is formed among the pump 213, the culture medium 212, the filtering chamber 204 and the collecting vessel 209 by closing valves located respectively at lines leading to the first inlet 204, the second inlet 205, the drain 207 and the optional storage chamber 210 (data not shown). In one example, the stem-cell culture medium is the StemSpan SFEM medium supplemented with a mixture of cytokines and low density lipoproteins (LDLs). The StemSpan SFEM medium is purchased directly from StemCell Technologies (USA), which does not contain hematopoietic growth factors or other cytokines. The mixture of cytokines includes a recombinant human stem cell factor, a recombinant human thrombopoietin and a recombinant human Flt-3 ligand. Each cytokine is present in a concentration between 5 ng/ml to 500 ng/ml at the beginning of the culture, and preferably in a concentration between 10 to 100 ng/ml at the beginning of the culture. The use of LDL is optional, and when LDL is included, it is usually present at a dose between 0.1 mg/ml to 20 mg/ml in the beginning of the culture, and preferably at 5 mg/ml in the beginning of the culture. The culture medium 212 are replaced by fresh medium 212 every other day, aliquots of the spent medium 212 are also sampled to determine the number of hematopoietic stem cells in the medium 212. Once the number of hematopoietic stem cells 202 in the sampling medium 212 has reached a predetermined value, then the medium 212 in the filtering chamber 211 is drained from the second outlet 208 and into the hematopoietic stem cell collecting vessel 209, and hematopoietic stem cells 202 are then harvested from the medium 212 in the collecting vessel 209. Alternatively, the hematopoietic stem cells 202 may also be continuously harvested every few days from the medium 212 collected in the collecting vessel 209 and then pool together for further use. In one example, hematopoietic stem cells that may be harvested form the medium 212 is at least one fold at day 1 after ex vivo expansion as compared with the control, i.e., stem cells isolated from the cord blood and then subjected to direct ex vivo culture, and may reached as high as 14 to 15 folds at day 10 after ex vivo expansion.

Furthermore, hematopoietic stem cells that are isolated, ex vivo expanded and harvested by the continuous system of this embodiment still possess the ability to form colony. In one example, the number of colonies formed by the isolated and expanded hematopoietic stem cells is relatively the same as that in the control, about 150 colonies are counted (data not shown).

Similarly, the continuous-type system of this embodiment is an easy to use and time efficient system for isolating hematopoietic stem cells, the operating time starting from permeating the source of hematopoietic stem cells into the filtering chamber to subjecting the isolated hematopoietic stem cells to ex vivo culture in the system, that is, without taking into account the time required for ex vivo culture, takes a relatively short period of time, from about 10 min to about 60 min. In this embodiment, the operating time is about 18 min.

The following Examples are provided to illustrate certain aspects of the present invention and to aid those of skill in the art in practicing this invention. These Examples are in no way to be considered to limit the scope of the invention in any manner.

### EXAMPLES

### Example 1

### Preparation of Surface Modified PU Membrane

1.1 Preparation of PU-GMA membrane

PU-GMA membranes were prepared by a plasma discharge method described in Example 1 of a prior publication, JP 2005-323534, which is hereby incorporated by reference in its entirety. Briefly, a polyurethane membrane, such as the one taken from a leukocyte removal filter (Imguard III-RC, Terumo Corporation) is used in this example. Glycidyl methacrylate (GMA) was grafted onto the surface of the PU membrane in a reaction vessel with the aid of Ar plasma. The Ar plasma was generated by applying a high frequency power (˜200 Watts) (Adtec Co., AX-300) to a flow of Ar gas (at a pressure of about 26.6 Pa) in the reaction vessel for about 30 sec, and graft polymerization between GMA and the PU membrane was then performed in the reaction vessel for 5 min at a pressure of about 0.65 Pa. Subsequently, the PU membranes grafted with GMA was washed with mixed solution of water and methanol (1:1 volume ratio) under shaking for 30 min, and with ultra pure water for another 30 min. The degree of GMA being introduced onto the surface of PU membranes was determined by the following equation:

Ratio of GMA introduction (%)=(X/Y)×100

In which the ratio of GMA introduction was defined as the ratio of the dry weight of PU-GMA membranes (vacuum dry at 80° C. for 24 hr) after plasma discharge (Xg) divided by the dry weight of PU membranes (vacuum dry at 80° C. for 24 hr) before plasma discharge (Yg). In this example, the ratio was about 0.61%.
1.2 Preparation of PU—COOH membranes PU-GMA membrane (pore size estimated to be about 5 μm) prepared by the procedures described above in example 1.1 was cut into circles; each was 25 mm in diameter. Three sheets of PU-GMA membranes were immersed in a glycine containing NaOH solution (20 ml, 0.1M, the concentration of glycine in NaOH solution is 0.4M), and incubated at 80° C. for 24 hrs so as to prepare PU membrane having carboxylic acid groups on its surface. After the reaction was completed, the membranes were washed in ultra pure water under vibration for 10 min at 25° C. The washing step was repeated twice, and the membranes were finally immersed in ultra pure water, and stored in refrigerator at 4° C. before use. PU membrane thus prepared is termed “PU—COOH membrane” hereafter.

### Example 2

### A Batch System for Isolating, Ex Vivo Expanding and Harvesting Hematopoietic Stem Cells

Umbilical cord blood was obtained from pregnant women with written informed consent. The cord blood was collected in a blood bag (CPDA-1 Termo Co.) containing anticoagulants such as citric acid, dextrose etc. A filtering device was constructed by fitting 3 sheets of PU—COOH membranes of example 1.2 onto a filter holder (25 mm in membrane diameter, Millipore Co.). 6 ml of umbilical cord blood was then permeated through the filtering device at a flow rate of 1 ml/min. Washing solution (6 ml) was then permeated through the filtering device at 1 ml/min for 6 min. Plasma A or StemSpam SFEM medium was used as a washing solution. Plasma A was prepared as follows: platelet-poor plasma was obtained by centrifuging umbilical cord blood at a speed of 1,800 rpm, and then filtered through a 0.22 μm disposable filter (Millex GS, Millipore Co.) to remove blood cells completely (Plasma A). StemSpam SFEM medium was directly purchased from the provider (#09650, StemCell Technologies Co.).

After washing, PU—COOH membranes were removed from the membrane holder and immersed into a culture medium, which will be described in details below, and hematopoietic stem cells adhered on PU-COOH membranes were cultured in a CO₂ incubator for 10 days at 5% CO₂ and 37° C. In this example, the culture medium used for ex vivo culturing of the isolated hematopoietic stem cells was prepared by supplementing StemSpan SFEM medium with cytokine cocktail of StemSpan CC110 (#02697, StemCell Technologies) and 5 mg/ml of low density lipoprotein (LDL), and the culture medium is hereafter called HSC medium A. The operating time of the whole procedures from permeation of umbilical cord blood through the membranes to the inoculation of hematopoietic stem cells to culture dishes is only 18 min and is found to be very short.

The number of hematopoietic stem cells was analyzed by flow cytometry (Beckman-Coulter Co., EPICS™ XL) after 10 days of culture. The analysis of CD34⁺ cells was conducted based on the protocol set forth in ISHAGE guideline (International Society of Hematotherapy and Graft Engineering, Keeney M. et al., Cytometry (1998) 34, 61-70). The same number of sample tubes (Beckman-Coulter Co.) was prepared to the number of samples. Twenty lo microliters of anti-CD34 antibody (Beckman-Coulter Co. CD45-FITC/CD34-PE) were added to each sample tubes containing samples. After 100 μL of each sample was added into each sample tube containing anti-CD34 antibody, the sample was well agitated followed by injection of 20 μl of cell viability dye (Beckman-Coulter Co., 7-AAD Viability Dye), and incubated under dark for 15 min at room temperature. Subsequently, 500 μL of lysing solution (OptiLyse C) was injected into each sample tube, mixed extensively and incubated for 10 min at room temperature. 500 μl of phosphate buffer saline (PBS) was added into each sample tube, agitated well, and incubated for 10 min at room temperature. Finally the prepared samples were analyzed by flow cytometry. CD34⁺ hematopoietic stem cell numbers in umbilical cord blood collected for this example was first analyzed and used as a reference for comparing the isolation effects of using plasma A or StemSpam SFE medium as a washing solution and on subsequent ex vivo expansion efficiency of CD34⁺ hematopoietic stem cells. The number of CD34⁺ hematopoietic stem cells in the culture medium after ex vivo expansion and harvest was analyzed. Ex vivo expansion ratio (EVER) of CD34⁺ hematopoietic stem cells was, therefore, calculated by the equation as follows:

EVER=N₂/N₁×100

in which N₂ represents the number of CD34⁺ hematopoietic stem cells in the culture medium after ex vivo expansion and harvest; and N₁ represent the number of CD34⁺ hematopoietic stem cell numbers in umbilical cord blood collected for the experiments. Results are provided in Table 1, in which expansion ratio of CD34⁺ hematopoietic stem cells increases for about 2.5 and 0.63 times respectively, for washing with plasma A and StemSpan SFEM medium.

TABLE 1
Ex vivo expansion ratio (EVER) of CD34⁺ hematopoietic stem cells by
use of plasma A or StemSpan SFEM medium as a washing solution

| | Plasma A | StemSpan SFEM medium |
|---|---|---|
| EVER (%) | 253 | 63.6 |

### Example 3

### Hematopoietic Stem Cells that were Isolated, Expanded and Harvested by the Batch System of Example 2

Hematopoietic stem cells were isolated, expanded ex vivo and harvested in accordance with the procedures described in Example 2, except 1 ml of umbilical cord blood was used instead of 6 ml of umbilical cord blood, and culture medium (i.e., HSC medium A) was and was not exchanged on day 5 after cell culture started. Plasma A was used as a washing solution in this experiment. Results are presented in Table 2, and an expansion ratio about 2.7-6.2 folds (as compared with control) is obtained for stems cells isolated and expanded in accordance with the method described in this example.

The operating time of the whole procedures from permeation of umbilical cord blood through the membranes to the inoculation of hematopoietic stem cells to culture dishes was less 15 min and is found to be very short.

TABLE 2
Ex vivo expansion ratio (EVER) of CD34⁺ hematopoietic
stem cells by use of plasma A as a washing solution with and
without changing culture medium

| | Culture medium unchanged | Culture medium changed |
|---|---|---|
| EVER (%) | 271 | 623 |

### Example 4

### A Continuous Systems for Isolating, Ex Vivo Expanding and Harvesting Hematopoietic Stem Cells

Umbilical cord blood was collected as described above in example 2. A continuous system for the isolation, ex vivo expansion and harvest of hematopoietic stem cells was set up as depicted in FIG. 2. 20 g of umbilical cord blood (in a blood bag) was fed through the filtering chamber 201 comprising therein 6 sheets of polyurethane (PU) membranes (Imugard III-RC, Terumo Co.) at a flow rate of 2 ml/min. The average pore size of each sheet of PU-membranes is between 5 to 12 μm, in which the pore size was measured by a capillary flow porometer (Porous Materials Inc.). The cord blood permeated through the filtering chamber was then stored in the storage chamber 206. Plasma A (20 g) was then permeated through the filtering chamber 201 at a flow rate of 2 ml/min, followed by StemSpan SFEM medium (60 g) to rinse the filtering chamber 201. Then, HSC medium A (i.e., StemSpan SFEM medium supplemented with cytokine cocktail of StemSpan CC110 (#02697 , StemCell Technologies) and 5 mg/ml of LDL) was fed through the filtering chamber 201, and circulated at a speed of 0.5 ml/min under the aid of a peristalic pump. Fresh HSC medium A (20 g) was introduced into the system on days 1 and 5, respectively, and the expent HSC medium A was drained out through the outlet 206 and was further led to the drain 207, while culturing of the retained hematopoietic stem cells continued in the filtering chamber 201. A small aliquot of HSC medium A was taken on days 1, 6 and 10, respectively, from the drain 207 to analyze the numbers of hematopoietic stem cells in the culture medium. The cell numbers were measured in accordance with the procedures described in Example 1. Results are presented in Table 3.

It is clear form results presented in Table 3 that the continuous system of this example provides a high expansion ratio of stem cells, at least 6.8 folds as compared with a control (i.e., stem cells taken from the cord blood and then directly subjected to conventional culturing procedures). Furthermore, the procedure from permeating the cord blood through the continuous system in this example until introducing HSC medium A to the filtering chamber for continue culturing, merely takes about 30 min, further demonstrating that this continuous system for separation, ex vivo expansion and harvest of stem cells is relatively easy to use compared with the known stem cells' isolating and/or culturing techniques.

TABLE 3
Ex vivo expansion ratio (EVER) of CD34⁺ hematopoietic stem cells
measured at various days during culture

| | Day 1 | Day 6 | Day 10 | Total. |
|---|---|---|---|---|
| EVR (%) | 179 | 375 | 130 | 684 |

### Example 5

### Hematopoietic Stem Cells that were Isolated, Expanded and Harvest by the Continuous System of Example 4

Hematopoietic stem cells were isolated, expanded ex vivo and harvested in accordance with the procedures described in Example 4, except culture medium (i.e., HSC medium A) was exchanged on days 2, 4, 6 and 8 after cell culture started. Again, the procedure from permeating the cord blood through the continuous system until introducing HSC medium A to the filtering chamber for continue culturing took about 30 min. Results are presented in Table 4, and an expansion ratio about 46 folds (as compared with control) is obtained for stems cells isolated and expanded in accordance with the method described in this example.

TABLE 4
Ex vivo expansion ratio (EVER) of CD34⁺ hematopoietic stem cells
measured at various days during culture

| | Day 2 | Day 4 | Day 6 | Day 8 | Day 10 | Total |
|---|---|---|---|---|---|---|
| EVR (%) | 547 | 168 | 1,104 | 1,281 | 1,468 | 4,567 |

### Example 6

### Isolation, Ex Vivo Expansion and Harvest of Hematopoietic Stem Cells in the Continuous System of Example 4 Using Non-Woven Fabric as a Filter

Hematopoietic stem cells were isolated, expanded ex vivo and harvested in accordance with the procedures described in Example 4, except non-woven fabric (Asahi Medical Co., SepaCell R, R-500B2(3)-1) was used as a filtering membrane instead of the polyurethane membranes, and culture medium (i.e., HSC medium A) was exchanged on days 2, 4, 6 and 8 after cell culture started.

Results are presented in Table 5, and an expansion ratio about 3.7 folds (as compared with control) is obtained for stems cells isolated and expanded in accordance with the method described in this example.

TABLE 5
Ex vivo expansion ratio (EVER) of CD34⁺ hematopoietic stem cells
measured at various days during culture

| | Day 2 | Day 4 | Day 6 | Day 8 | Day 10 | Total |
|---|---|---|---|---|---|---|
| EVR (%) | 1.1% | 2.4% | 136% | 167% | 65% | 371% |

### Example 7

### Isolation, Ex Vivo Expansion and Harvest of Hematopoietic Stem Cells From Peripheral Blood using the Continuous System of Example 4

Hematopoietic stem cells are isolated, expanded ex vivo and harvested in accordance with the procedures described in Example 4, except peripheral blood is used as a stem cell source instead of cord blood, and fresh culture medium (i.e., HSC medium A) are provided on days 2, 4, 6 and 8 after cell culture starts. It is expected to see an increase in the expansion ratio (as compared with control) for stems cells isolated and expanded in accordance with the method described in this example.

### Example 8

### Isolation, Ex Vivo Expansion and Harvest of Hematopoietic Stem Cells From Bone Marrow Aspirates using the Continuous System of Example 4

Hematopoietic stem cells are isolated, expanded ex vivo and harvested in accordance with the procedures described in Example 4, except bone marrow aspirates are used as a stem cell source instead of cord blood, and fresh culture medium (i.e., HSC medium A) are provided on days 2, 4, 6 and 8 after cell culture starts. It is expected to see an increase in the expansion ratio (as lo compared with control) for stems cells isolated and expanded in accordance with the method described in this example.

### Comparative Example

Mononuclear cells were isolated from umbilical cord blood by using conventional Ficoll-Paque method (See Fotino M., et al., Ann. Clin. Lab. Sci., (1971) 1:131-133). Briefly, umbilical cord blood was diluted in a ratio of 1:4 (v/v) with phosphate buffer saline (PBS) containing 2 mmol/L EDTA and 0.5% bovine serum albumin (BSA) (hereafter the PBS is termed “PBS A”). 35 ml of the diluted umbilical cord blood was then added onto the surface of 15 ml of Ficoll-Paque solution (Pharmacia), and then subjected to centrifugation (400×g for 40 min at 20° C.). After centrifugation, the separated mononuclear layers were carefully isolated. PBS A was then added into the mononuclear solution until a total volume of 50 ml was reached. Subsequently, the solution was agitated, and centrifuged again at 300×g for 10 min.

Supernatant of the solution was removed, leaving behind 0.5 ml of bottom solution. The hematopoietic stem cell solution that prepared contained 5,181 cells/μl of red blood cells, 11,935 cells/μl of white blood cells, 171,812 cells/μl of platelets, and 185 cells/μl of CD34⁺ hematopoietic stem cells analyzed in accordance with ISHAGE guideline. The entire procedures took about 3 hrs to complete.

1,000 cells of CD34⁺ hematopoietic stem cells purified by the procedures described above were inoculated into 24 well plates containing 1 ml of culture medium for hematopoietic stem cell (HSC medium A). Ex vivo expansion ratio (EVER) of hematopoietic stem cells in the culture medium was analyzed in according to the same method described in Example 2, EVER was 143% after 10 days culture.

In conclusion, the processing time for isolating hematopoietic stem cells in the conventional Ficoll-Paque method was much longer than the method shown in Examples 2, 3, 4, 5, and 6 of this invention. The ex vivo expansion ratio (EVER) of CD34⁺ hematopoietic stem cells using CD34⁺ cells isolated by Ficoll-Paque method was less than that by the method of this invention as shown in Examples 3, 4, 5 and 6.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features. From the above description, one skilled in the art can easily ascertain the essential characteristics of the present invention, and without departing from the spirit and scope thereof, can make various changes and modifications of the invention to adapt it to various usages and conditions. Thus, other embodiments are also within the scope of the following claims.

1. A system for isolating, ex vivo expanding and harvesting hematopoietic stem cells, comprising:
a filtering chamber comprising a membrane having a pore size ranges from 2 pm to 100 pm;
a first inlet for introducing a source of hematopoietic stem cells into the filtering chamber;
a second inlet for introducing a washing solution into the filtering chamber; and
a first outlet for draining the washing solution out of the filtering chamber.

2. The system of claim 1, further comprising a storage chamber for storing the source of hematopoietic stem cells after it has been permeated through the filtering chamber.

3. The system of claim 1, wherein the washing solution is any of a serum-free culture medium, a serum-containing culture medium, a saline, a buffer solution, an EDTA containing saline, an EDTA containing buffer solution, a platelet-poor plasma or combinations thereof.

4. The system of claim 1, wherein the source of hematopoietic stem cells is any of a cord blood, a bone marrow aspirates or a peripheral blood.

5. The system of claim 1, wherein the hematopoietic stem cells are retained by the membrane.

6. The system of claim 1, wherein the membrane is a polyurethane- based polymer or a polyethylene terephthalate-based polymer.

7. The system of claim 6, wherein the polyethylene terephthalate-based polymer further comprises a polymer coating made from at least one monomer selected from the group consisting of hydroxyethyl methacrylate (HEMA), dimethylaminoethyl methacrylate (DM), n-butylmethacrylate (BMA), N,N-dimethylacrylamide (DMA), N-acryloylmorpholine (AMO) and N-vinylpyrrolidone (VP).

8. The system of claim 1, wherein the operating time for the system is between about 10 min to about 30 min.

9. The system of claim 1, further comprising:
a third inlet for introducing a stem-cell culture medium into the filtering chamber;
a pump for circulating the stem-cell culture medium in the system; and
a second outlet for collecting the hematopoietic stem cells.

10. The system of claim 9, further comprising a storage chamber for storing the source of hematopoietic stem cells after being permeating through the filtering chamber.

11. The system of claim 9, wherein the stem-cell culture medium further comprises a cytokine

12. The system of claim 11, wherein the stem-cell culture medium further comprises a low density lipoprotein.

13. The system of claim 9, wherein the hematopoietic stem cells are harvested from the stem-cell culture medium collected from the second outlet.

14. A method for isolating, ex vivo expanding and harvesting hematopoietic stem cells, comprising in sequence the steps of:
(a) providing a system of claim 1;
(b) introducing the source of hematopoietic stem cells form the first inlet into the filtering chamber;
(c) introducing the washing solution from the second inlet into the filtering chamber;
(d) culturing the membrane in a stem-cell culture medium to expand the hematopoietic stem cells retained therein.

15. The method of claim 14, further comprising (c1) draining the washing solution out of the filtering chamber from the first outlet after step (c).

16. The method of claim 14, wherein the source of hematopoietic stem cells is any of cord blood, bone marrow aspirates or peripheral blood.

17. The method of claim 14, wherein the washing solution is any of a serum-free culture medium, a serum-containing culture medium, a saline, a buffer solution, an EDTA containing saline, an EDTA containing buffer solution, a platelet-poor plasma or combinations thereof.

18. The method of claim 14, wherein the membrane is a polyurethane-based polymer or a polyethylene terephthalate-based polymer.

19. The method of claim 18, wherein the polyethylene terephthalate-based polymer further comprises a polymer coating made from at least one monomer selected from the group consisting of hydroxyethyl methacrylate (HEMA), dimethylaminoethyl methacrylate (DM), n-butylmethacrylate (BMA), N,N-dimethylacrylamide (DMA), N-acryloylmorpholine (AMO) and N-vinylpyrrolidone (VP).

20. A method for isolating, ex vivo expanding and harvesting hematopoietic stem cells, comprising in sequence the steps of:
(a) providing a system of claim 9;
(b) introducing the source of hematopoietic stem cells form the first inlet into the filtering chamber;
(c) introducing the washing solution from the second inlet into the filtering chamber;
(d) introducing the stem-cell culture medium from the third inlet into the filtering chamber;
(e) activating the pump to circulate the stem-cell culture medium in the system; and
(f) harvesting the hematopoietic stem cells from the stem-cell culture medium collected from the second outlet.

21. The method of claim 20, further comprising:
(b1) storing the source of hematopoietic stem cells in a storage chamber after it has been permeated through the filtering chamber in step (b);
(c1) draining the washing solution out of the filtering chamber from the first outlet after step (c); and
(e1) sampling the hematopoietic stem cells by taking an aliquot of the stem-cell culture medium from the second outlet after step (e).

22. The method of claim 20, wherein the source of hematopoietic stem cells is any of a cord blood, bone marrow aspirates or a peripheral blood.

23. The method of claim 20, wherein the washing solution is any of a serum-free culture medium, a serum-containing culture medium, a saline, a buffer solution, an EDTA containing saline, an EDTA containing buffer solution, a platelet-poor plasma, or a combinations thereof.

24. The method of claim 20, wherein the membrane is a polyurethane-based polymer or a polyethylene terephthalate-based polymer.

25. The method of claim 24, wherein the polyethylene terephthalate-based polymer further comprises a polymer coating made from at least one monomer selected from the group consisting of hydroxyethyl methacrylate (HEMA), dimethylaminoethyl methacrylate (DM), n-butylmethacrylate (BMA), N,N-dimethylacrylamide (DMA), N-acryloylmorpholine (AMO) and N-vinylpyrrolidone (VP).
